(19) United States
(12) Patent Application Publication
(10) Pub. No.: US 2026/0033888 A1
(43) Pub. Date: Feb. 5, 2026

(54) CURVED FIRE-FORWARD NEEDLE FOR PUNCTURING AND TRAVERSING VASCULATURE

(71) Applicant: BARD PERIPHERAL VASCULAR, INC., Franklin Lakes, NJ (US)

(72) Inventors: Kathryn BLANK, Chandler, AZ (US); Alex BRODERICK, Chandler, AZ (US); Summer Lindsay FORD, Queen Creek, AZ (US); Alexander PALMER, Gilbert, AZ (US); Samuel Michael SHREVE, Scottsdale, AZ (US)

(73) Assignee: BARD PERIPHERAL VASCULAR, INC., Franklin Lakes, NJ (US)

(21) Appl. No.: 18/995,740

(22) PCT Filed: Aug. 18, 2022
(86) PCT No.: PCT/US2022/075131

§ 371 Date: Jan. 16, 2025

(51) Int. Cl. A61B 18/14; A61B 18/00

(52) U.S. Cl. CPC A61B 18/1477; A61B 2018/00184; A61B 2018/00291; A61B 2018/00428; A61B 2018/00601; A61B 2018/1425; A61B 2018/1475; A61B 2218/007

(57) ABSTRACT

A needle firing assembly of methods of use includes a firing device, a catheter extending from the firing device, a needle extending from the firing device and through an interior of the catheter, the needle having a curvature proximate a distal end of the needle opposite the firing device, and a suction device in fluid communication with an interior of the catheter. The suction device is configured to draw a suction force through a distal end of the catheter opposite the firing device. The firing device is configured to move the needle in a forward firing direction relative to the catheter to extend the distal end of the needle through the distal end of the catheter.

### TECHNICAL FIELD

The present specification generally relates to needle firing assemblies and methods of use and, more specifically, needle firing assemblies including curved needles for creating a passageway for stent placement between adjacent vascular entities.

### BACKGROUND

Puncturing an exterior or interior surface of a vasculature wall may be challenging due to the slippery and unstable nature of vessels. As such, aortorenal bypass procedures may be performed in an open surgery manner to ensure that adjacent vessels are punctured accurately when placing a stent.

Accordingly, a need exists for improved devices that permit a needle to be passed through adjacent vascular bodies so that a stent may be accurately placed when performing aortorenal bypass procedures in less invasive manners.

### SUMMARY

In one embodiment, a needle firing assembly includes: a firing device; a catheter extending from the firing device; a needle extending from the firing device and through an interior of the catheter, the needle having a curvature proximate a distal end of the needle opposite the firing device; and a suction device in fluid communication with an interior of the catheter, the suction device configured to draw a suction force through a distal end of the catheter opposite the firing device, wherein the firing device is configured to move the needle in a forward firing direction relative to the catheter to extend the distal end of the needle through the distal end of the catheter.

In another embodiment, a needle firing assembly includes: a firing device; a catheter extending from the firing device; a needle extending from the firing device and through an interior of the catheter; and a suction device in fluid communication with an interior of the catheter configured to draw fluid through a distal end of the catheter, wherein the firing device is configured to move the needle relative to the catheter to extend a distal end of the needle through the distal end of the catheter when in a firing state and retract the needle relative to the catheter such that the distal end of the needle is withdrawn from the distal end of the catheter when in a retracted state.

In yet another embodiment, a method for puncturing a surface of a vascular entity includes: positioning a needle firing assembly relative to a surface of a vascular entity. The needle firing assembly includes: a firing device; a catheter extending from the firing device; a needle extending from the firing device and through an interior of the catheter, the needle having a curvature proximate a distal end of the needle, a distal end of the catheter positioned at the surface of the vascular entity; and a suction device in fluid communication with an interior of the catheter. The method further includes: operating the suction device to draw fluid through the distal end of the catheter toward the firing device; creating a suction force drawing the surface of the vascular entity toward the distal end of the catheter; and operating the firing device to move the needle in a forward firing direction relative to the catheter to extend the distal end of the needle through the distal end of the catheter and puncture the surface of the vascular entity.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a side view of an embodiment of a needle firing assembly, according to one or more embodiments shown and described herein;

FIG. 2 schematically depicts a cross-section view of a firing device of the needle firing assembly of FIG. 1;

FIG. 3 schematically depicts a side view of another embodiment of a needle firing assembly, according to one or more embodiments shown and described herein;

FIG. 4 schematically depicts a side view of yet another embodiment of a needle firing assembly, according to one or more embodiments shown and described herein;

FIG. 5 schematically depicts a side view of one other embodiment of a needle firing assembly, according to one or more embodiments shown and described herein;

FIG. 6 schematically depicts a perspective view of a needle of the needle firing assembly of FIG. 1 extending through a first vascular entity and a second vascular entity;

FIG. 7 schematically depicts a side view of yet another embodiment of a needle firing assembly, according to one or more embodiments shown and described herein;

FIG. 8 schematically depicts a cross-section view of a catheter and a needle of the needle firing assembly of FIG. 7 taken along line 8-8 of FIG. 7;

FIG. 9 schematically depicts a side view of another embodiment of a needle firing assembly, according to one or more embodiments shown and described herein;

FIG. 10 schematically depicts a partial side view of the needle firing device of either of FIGS. 7 and 9 in a first position positioned adjacent a surface of a vascular entity;

FIG. 11 schematically depicts a partial side view of the needle firing device of either of FIGS. 7 and 9 in a second position drawing a suction force into a suction channel of the catheter to position a distal end of the catheter adjacent the surface of the vascular entity; and

FIG. 12 schematically depicts a partial side view of the needle firing device of either of FIGS. 7 and 9 in a third position drawing a suction force into a suction channel and firing the needle in a forward firing direction into the vascular entity.

### DETAILED DESCRIPTION

Embodiments described herein are directed to a needle firing assembly including a curved needle that may be fired into adjacent vascular entities to facilitate placement of a stent between the vascular entities. The curved needle of the present disclosure may aid in an endovascular or percutaneous, minimally invasive procedure rather than in an open surgery manner to place a stent between vascular entities, as described herein, as the needle curvature aids to create a path for placing the stent to be positioned between adjacent vessels. Various embodiments of the needle firing assembly and the operation of the needle firing assembly are described in more detail herein. Whenever possible, the same reference numerals will be used throughout the drawings to refer to the same or like parts.

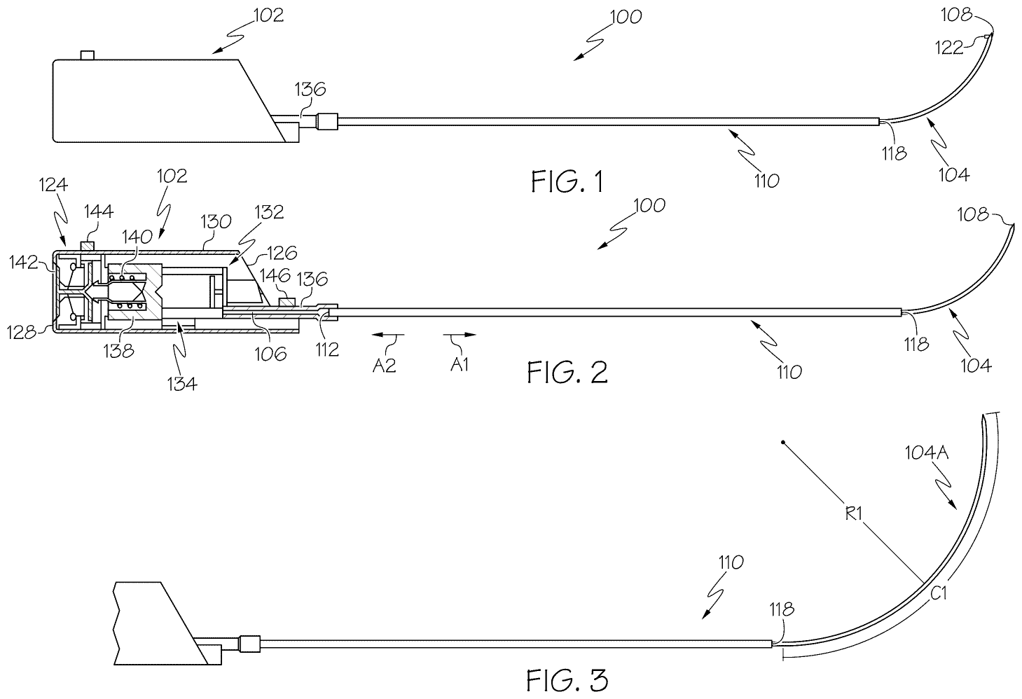

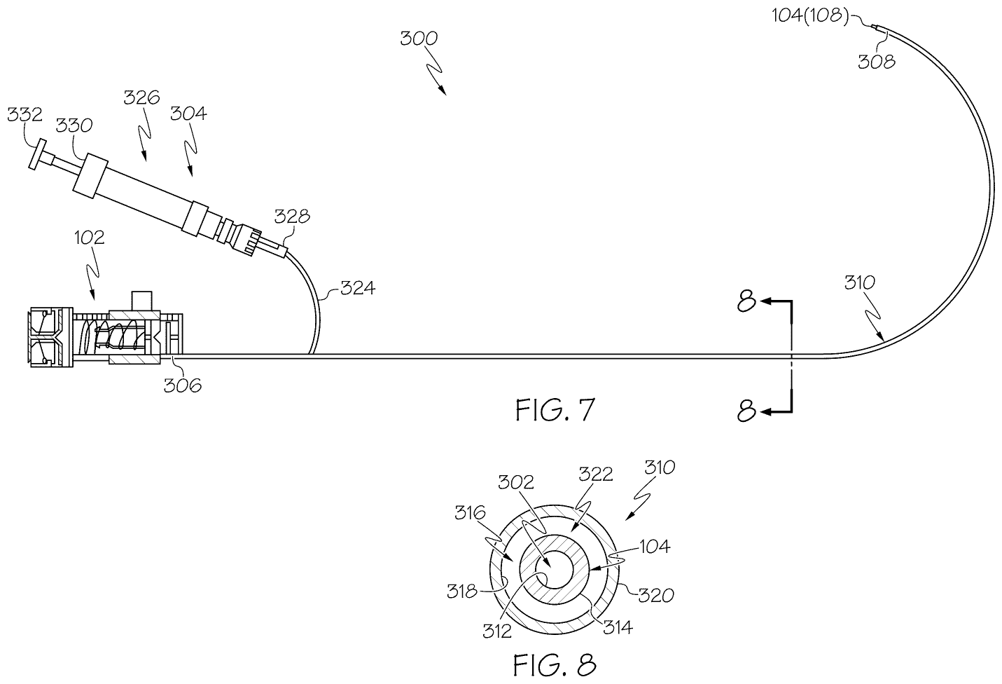

Referring now to FIG. 1, a needle firing assembly 100 is illustrated according to one or more embodiments described herein. The needle firing assembly 100 generally includes a firing device 102 and a needle 104 engaging the firing device 102. Embodiments of the needle firing assembly 100 as described herein further generally include a catheter 110 extending from the firing device 102, the needle 104 extending from the firing device 102 and through an interior of the catheter 110. As described in greater detail further below with respect to FIGS. 7-12, embodiments of the needle firing assembly 100 as described herein may further include a suction device 304 in fluid communication with the interior of the catheter 110. 310. The needle 104 has a curvature proximate a distal end 108 of the needle 104 opposite the firing device 102. The suction device 304 is configured to draw a suction force through a distal end 118 of the catheter 110 opposite the firing device 102. The firing device 102 is configured to move the needle 104 in a forward firing direction (e.g., distal direction) relative to the catheter 110 to extend the distal end 108 of the needle 104 through the distal end 118 of the catheter 110.

As shown in FIGS. 1-2, the needle 104 has a proximal end 106 and a distal end 108. In embodiments, the needle 104 includes a first needle portion that is substantially linear and a second needle portion that has a curvature formed therein. The distal end 108 of the needle 104 may define a tapered edge for facilitating puncturing through a vascular entity, as described in more detail herein. Although not shown, the needle 104 has a hollow interior through which a guidewire may be directed for placement of a stent graft after the needle 104 is used as described herein to create a passageway between a pair of punctured vascular entities. In embodiments, the needle 104 further includes one or more puncture assist devices 122 provided at the distal end 108 of the needle 104 to assist puncturing the needle 104 through a vascular entity. In embodiments, the puncture assist device 122 may include one or more of a radiofrequency (RF) cutting device, a laser, and other suitable mechanisms to aid in puncturing a vascular entity. The puncture assist device 122 may be activated either manually or automatically upon contact with a surface of a vascular entity.

The curvature of the second needle portion of the needle 104 proximate the distal end 108 has a radius of curvature and is defined by a circumferential length. As referred to herein, the radius of curvature of the second needle portion of the needle 104 refers to the radius of a circular arc defining the curvature of the second needle portion. As the needle 104 that is rigid is fired distally by the firing device 102, the distal end 108 of the needle 104 distally advances past the distal end 118 of the catheter 110, which is flexible and which conforms to the shape of the needle against which it is disposed in any given position. Thus, FIGS. 1-6 show the needle 104, 104A, 104B, 104C in a fired position. In other embodiments, the needle 104 may be formed from a semi-rigid material such as, for example, nickel titanium (NiTi), and can be straightened when positioned within the catheter, i.e., in a retracted position, which is formed from a material having a rigidity greater than a rigidity of the needle 104. Thus, when the needle 104 distally advances past the distal end 118 of the catheter 110, the second needle portion of the needle 104 assumes the particular radius of curvature.

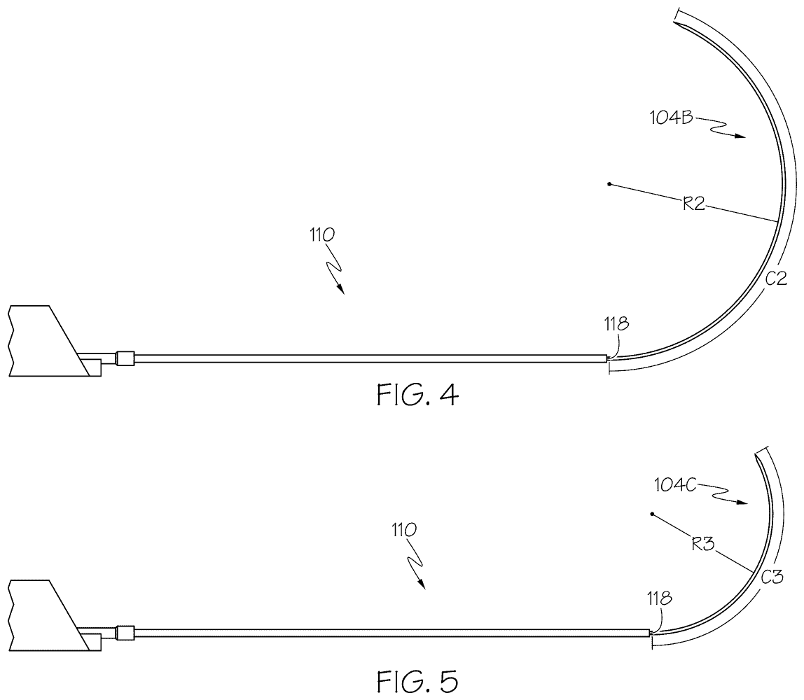

It should be appreciated that the needle 104 may include any suitable curvature based upon the procedure to be performed. The specific parameters of the curvature of the second needle portion may be based on, for example, a distance between two vascular entities through which the needle 104 is to be inserted, a size of the vascular entities, and the like. Accordingly, as shown in FIGS. 3-5, a plurality of needles are illustrated each including a second needle portion of the needle 104 having a varying curvature. Specifically, a needle 104A is illustrated in FIG. 3 having a first curvature defining a first circumferential length C1 and a first radius of curvature R1. A needle 104B is illustrated in FIG. 4 having a second curvature defining a second circumferential length C2 and a second radius of curvature R2. Although the second radius of curvature R2 of the needle 104B is different from the first radius R1 of the needle 104A, as shown, and the second circumferential length C2 of the needle 104B is greater than the first circumferential length C1 of the needle 104A. In embodiments, the needles 104A, 104B may include the same radius of curvatures R1, R2 and different circumferential lengths C1, C2, or vice versa. Additionally, a needle 104C is illustrated in FIG. 5 having a curvature defining a third circumferential length C3 and a third radius of curvature R3. As shown, the third circumferential length C3 may be less than the first circumferential length C1 and the second circumferential length C2, and the third radius R3 may be less than the first radius R1 and the second radius R2. It should be appreciated that these are merely non-limiting examples of various needles that may be used and the scope of the present disclosure is not limited to needles having such curvatures. Such curvature designs of the embodiments of the needle 104 described herein provide respective needle trajectories for conduit placement and offer varying curvature lengths (e.g., circumferential lengths) and radii of curvature to accommodate various conduit lengths and pathways for conduit placement.

Referring now to FIG. 2, a cross-sectional view of the firing device 102 is shown to better illustrate the internal components of the firing device 102. In embodiments, the firing device 102 generally includes a body 124 having a first surface 126, an opposite second surface 128, and one or more sidewalls 130 extending between the first surface 126 and the second surface 128. The first surface 126, the second surface 128, and the sidewall 130 define an interior 132 of the body 124 of the firing device 102. The firing device 102 may include an actuation assembly 134 for projecting the needle 104 as described herein. The actuation assembly 134 of FIG. 2 includes a receiver 136 extending through the first surface 126 of the body 124 for receiving the needle 104 and a proximal end 112 of the catheter 110, and specifically the proximal end 106 of the needle 104 proximally ending past the proximal end 112 of the catheter 110. As described in more detail herein, the needle 104 may be removed from the receiver 136. For example, in embodiments, the needle 104 may be press-fit proximally within the receiver 136 to be retained therein and removed by pulling the needle 104 from the receiver 136. In other embodiments, a fastener or fixing member may be provided to releasably retain the needle 104 proximally within the receiver 136.

In embodiments, the receiver 136 may be translatable through the first surface 126 of the body 124 of the firing device 102 in a forward firing direction A1 (e.g., distal direction) and an opposite rearward firing direction A2 (e.g., proximal direction). In embodiments, the actuation assembly 134 further includes a release member 138 provided within the interior 132 of the body 124 and fixedly coupled to the receiver 136, a biasing member 140 provided between the second surface 128 and the release member 138, and a trigger 142 accessible through the second surface 128 of the body 124 of the firing device 102. The trigger 142 may be depressed to engage the release member 138 and permit the biasing member 140 to move the release member 138 in a distal direction to distally press against and move the receiver 136 in the forward firing direction A1. In embodiments, upon the trigger 142 being reset or through another retraction mechanism coupled to the release member 138 and the biasing member 140, the biasing member 140 may proximally advance back in the rearward firing direction A2 to allow the release member 138 to return and draw the receiver 136 back. In embodiments, the actuation assembly 134, and the trigger 142, may be adjustable to adjust an advancement and/or retraction length of the needle 104. For example, a ratchet mechanism 144 may be provided to extend through the sidewall 130 of the body 124 of the firing device 102 to control a total distance that the release member 138 may travel upon actuation of the trigger 142. In embodiments, the ratchet mechanism 144 may be slidably positionable along the sidewall 130 to move between the forward firing direction A1 and the rearward firing direction A2. As a non-limiting embodiment, movement of the ratchet mechanism 144 in the forward firing direction A1 may increase the total distance in which the receiver 136 may be fired. Alternatively, movement of the ratchet mechanism 144 in the rearward firing direction A2 may decrease the total distance in which the receiver 136 may be fired. Additionally or alternatively, moving the ratchet mechanism 144 in the rearward firing direction A2 may facilitate drawing the receiver 136 back into the retracted position.

As such, the firing device 102 is operable between a (proximally) retracted state and a (distally) firing state with respect to the needle 104. When the firing device 102 is in the retracted state, the release member 138 and the receiver 136 are biased into the rearward firing direction A2 and the needle 104 provided within the receiver 136 is held in a retracted position relative to the firing device 102. When the firing device 102 is in the firing state, such as when the trigger 142 is depressed, the release member 138 and the receiver 136 are distally moved in the forward firing direction A1 and the needle 104 is distally moved from the retracted position to a firing position relative to the firing device 102 in the forward firing direction A1. In embodiments, after the firing device 102 is operated into the firing state, the firing device 102 may return, either automatically or manually, to the retracted state to move the needle 104 in the rearward firing direction A2 to return the needle 104 to the retracted position. It should be appreciated that other embodiments of the firing device 102 are within the scope of the present disclosure. Accordingly, other embodiments may include a partially or fully automated firing device including electrical, tactile buttons for directing the needle 104 in the forward firing direction A1 and returning the needle 104 in the reverse firing direction A2. Similarly, embodiments of the firing device 102 are contemplated in which the firing device 102 may be repeatedly actuated to project the needle 104 any number of times in a short succession of time. In embodiments, an adjustment mechanism 146 such as, for example, a wheel, dial, or the like, may be provided on the receiver 136 proximate the first surface 126 of the body 124 of the firing device 102. The adjustment mechanism 146 may be operated to rotate the catheter 110 and the needle 104 so as to change an angle or direction at which the needle 104 extends relative to the firing device 102. Additionally, a visual identifier such as, for example, a notch, marking, or other indicia, may be provided on the adjustment mechanism 146 to identify a direction in which the distal end 108 of the needle 104 extends relative to the firing device 102 since the distal end 108 of the needle 104 will not be visible when in use.

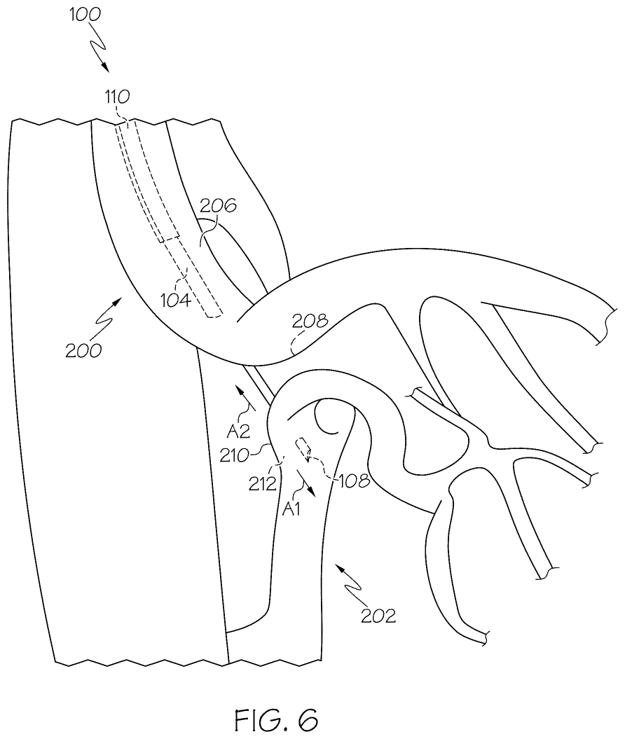

Referring now to FIG. 6, the needle firing assembly 100 is shown in use. Specifically, the needle 104 of the needle firing assembly 100 is illustrated extending through a first vascular entity 200 such as, for example, a hepatic vein, and a second vascular entity 202 such as, for example, a portal vein, as illustrated. However, it should be understood that the present disclosure is equally applicable to any vascular entities such as, for example, an aorta and renal artery. By extending the needle 104 through an interior and side wall of the first vascular entity 200 and through one side wall of the second vascular entity 202, a stent may be placed along a path created by the needle 104 between the first vascular entity 200 and the second vascular entity 202. As shown, the catheter 110 may be provided to surround at least a portion of the needle 104 to shield the portion of the needle 104 not distally projected from the catheter 110. The catheter 110 may extend from the firing device 102 (FIG. 1) along a portion of a length of the needle 104 while permitting the distal end 108 of the needle 104 to remain exposed after being distally projected. In other embodiments, prior to firing by the firing device 102, the catheter 110 may extend to the distal end 108 end of the needle 104. The distal end 108 of the needle 104 may be exposed when the firing device 102 is operated in the firing state to project the needle 104 from the catheter 110 and distally past the distal end 118 of the catheter 110. In embodiments, the catheter 110 may be formed from a flexible bio-compatible material such as extruded silicon tubing to conform to the curvature of the needle 104 extending through the catheter 110. In other embodiments, as noted hereinabove, the catheter 110 may be formed from any suitable material such as, for example, a braided or coiled polymer material, having a rigidity greater than a rigidity of the needle 104 such that the needle 104 would straighten when retracted within the catheter 110 and within the retracted position to allow for better maneuverability to a desired puncture location.

In use, the needle 104 is initially positioned within an interior of the first vascular entity 200 between an exterior surface 206 and an interior surface 206. The distal end 108 of the needle 104 is then positioned at and against the interior surface 208 of the first vascular entity 200 such that the distal end 108 of the needle 104 contacts the interior surface 208 of the first vascular entity 200. The needle 104 may be advanced by the firing device 102 as described herein and thus inserted through the interior surface 208 of the first vascular entity 200 and through an opposite exterior surface 206 of the first vascular entity 200 to emerge out of the first vascular entity 200. Through the distal advancement, the needle 104 may further be advanced through the second vascular entity 202 to puncture an exterior surface 210 of the second vascular entity 202 and be extended to puncture through an opposite interior surface 212 of the second vascular entity 202 as shown in FIG. 6. Accordingly, the needle 104 punctures through the first vascular entity 200 and partially through the second vascular entity 202 to define a pathway extending between the first vascular entity 200 and the second vascular entity 202. As described herein, the firing device 102 may be operated into the firing state to apply a distal force directing the needle 104 in the forward firing direction A1 to puncture the first vascular entity 200 and/or the second vascular entity 202.

As discussed herein, the needle 104 has a hollow interior through which a guidewire may extend along for positioning a stent. Once the needle 104 is positioned to extend through each of the first vascular entity 200 and the second vascular entity 202, the needle 104 may be detached from the firing device 102, specifically the receiver 136 of the firing device 102, to expose the proximal end 106 of the needle 104. This allows for a guidewire to be inserted into the hollow interior of the needle 104 and navigated through the needle 104 toward the distal end 108 of the needle 104. In other embodiments, a through hole may be formed in the firing device 102 to extend through the receiver 130 and the opposite second surface 128. The guidewire may be navigated through the through hole to be inserted through the needle 104 without requiring the needle 104 to be detached from the firing device 102. Once the guidewire has been navigated through the interior of the needle 104 and within the path formed by the needle 104 between the first vascular entity 200 and the second vascular entity 202, a stent may be positioned within the interior of the needle 104 between the needle 104 and the guidewire. After the stent is also positioned in the path formed by the needle 104 between the first vascular entity 200 and the second vascular entity 202, the needle 104 may be removed and the stent may be secured in position in any suitable manner.

It should be appreciated that a vascular entity such as, for example, the exterior surface 206, 210 or the interior surface 208, 212 of the respective first and second vascular entities 200, 202, may be slippery such that it is difficult to puncture a needle through the vascular entity. For example, when the needle 104 is projected forward by the firing device 102, the needle 104 may slip away from the vascular entity and miss an intended puncture point in the surface thereof. As such, it may be desirable to draw the surface of the vascular entity toward the needle 104 to create better contact between the needle 104 and the surface of the vascular entity prior to firing the needle 104. As discussed herein, this may be done by drawing a fluid, such as air, around the needle 104 in the rearward firing direction A2 to draw a portion of the surface of the vascular entity toward the needle 104 prior to firing and maintaining a position of the surface of the vascular entity during firing.

Referring now to FIG. 7, an embodiment of a needle firing assembly 300 is illustrated. The needle firing assembly 300 includes the same structure as the needle firing assembly 100, with the addition of a catheter 310, similar to the catheter 110, surrounding the needle 104, and a suction device 304 in fluid communication with an interior of the catheter 310. As such, like structure will be referred to herein with like reference numerals. The catheter 310 has a proximal end 306 and an opposite distal end 308. In embodiments, the proximal end 306 of the catheter 310 may extend from the firing device 102 and be removably secured thereto so that the firing device 102 may be readily removed from the catheter 310 during a procedure. In embodiments, a hermetic seal and/or valve may be provided at the proximal end 306 of the catheter 310 for sealing with the firing device 102.

As shown in FIG. 8, and discussed herein, the needle 104 has a hollow interior 302 defined by an interior surface 312 of the needle 104 opposite an exterior surface 314 of the needle 104. Similarly, the catheter 310, which surrounds the needle 104, has a hollow interior 316 defined by an interior surface 318 of the catheter 310 and an opposite exterior surface 320 of the catheter 310. The catheter 310 is a flexible member capable of bending along the curvature of the needle 104. As such, the needle 104 extends through the hollow interior 316 of the catheter 310 and along a length of the catheter 310 between the proximal end 306 of the catheter 310 and the distal end 308 of the catheter 310. The interior surface 318 of the catheter 310 is spaced apart from the exterior surface 314 of the needle 104 to define a suction channel 322.

Referring again to FIG. 7, the suction device 304 is in fluid communication with the hollow interior 316 of the catheter 310. A tube 324 extends from an end of the suction device 304 to the catheter 310, placing the suction device 304 in fluid communication with the suction channel 322 of the catheter 310. The suction device 304 is configured to create a suction force within the suction channel 322 when operated. As shown in FIG. 7, the suction device 304 is depicted as a syringe 326 having a first end 328 and an opposite second end 330. The tube 324 extends from the first end 328 of the syringe 326, and the syringe 326 includes a plunger 332 provided at the second end 330 thereof. In embodiments, the tube 324 may be joined to the first end 328 of the syringe 326 using a universal luer lock. The tube 324 is joined to the catheter 310 between the distal end 308 of the catheter 310 and the firing device 102. Upon drawing the plunger 332 of the syringe 326 in a direction opposite the first end 328, a suction force is drawn within the suction channel 322 between the catheter 310 and the needle 104. As discussed in more detail herein, the suction force draws a surface of a vascular entity against which the distal end 118 of the catheter 310 may be positioned toward the distal end 308 of the catheter 310. Alternatively, when the plunger 332 is released and/or a force is applied on the plunger 332 in a distal direction toward the second end 330 and toward the tube 324, suction within the catheter 310 is discontinued. As such, the surface of the vascular entity is no longer drawn toward the distal end 308 of the catheter 310.

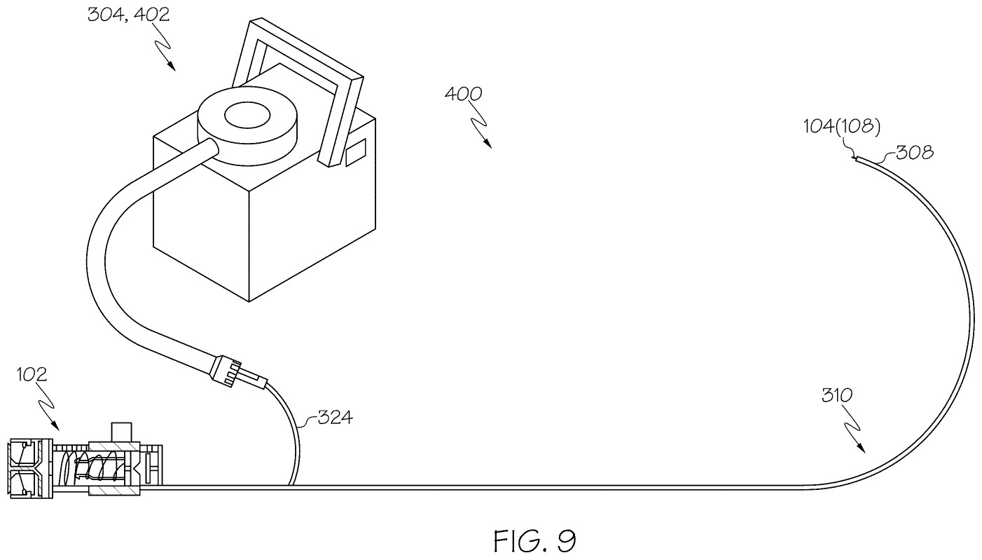

It should be appreciated that the suction device 304 may be any suitable device for drawing a suction force between the needle 104 and the catheter 310. For example, as shown in FIG. 9, an embodiment of a needle firing assembly 400 is illustrated including an external vacuum motor 402 as the suction device 304, such as a pump or the like, as opposed to the syringe 326. Similarly, the tube 324 extends from the external vacuum motor 402 to the catheter 310 and, in some embodiments, from a luer lock at the external vacuum motor 402. Accordingly, when the external vacuum motor 402 is activated, a suction force is drawn within the suction channel 322 provided between the catheter 310 and the needle 104 to create a suction within the suction channel 322, thereby proximally drawing the surface of the vascular entity toward the distal end 308 of the catheter 310. When the external vacuum motor 402 is deactivated, the suction force created within the suction channel 322 is discontinued and the surface of the vascular entity is no longer drawn toward the distal end 308 of the catheter 310.

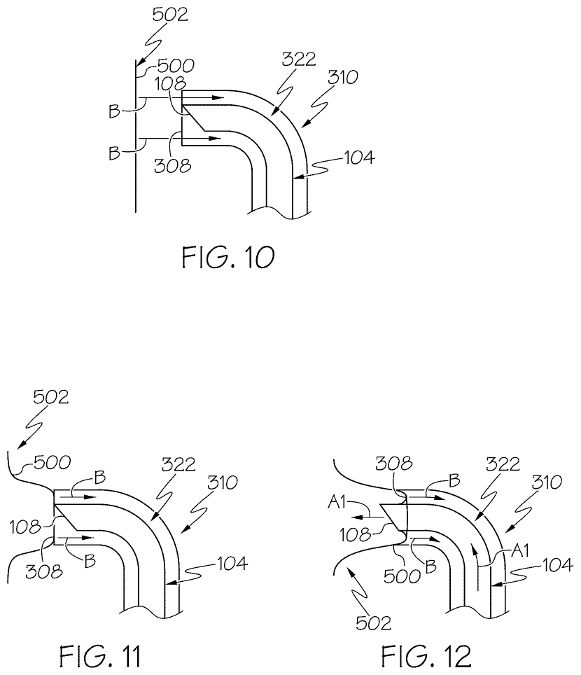

Referring now to FIGS. 10-12, operation of the needle firing assembly 300 is depicted in which the suction device 304 draws a portion of a surface of a vascular entity, such as the exterior surface 206, 210 and the interior surface 208, 212 of respective first and second vascular entities 200, 202, toward the distal end 308 of the catheter 310 and the distal end 108 of the needle 104.

More specifically, as shown in FIG. 10, the distal end 108 of the needle 104 is positioned at the distal end 308 of the catheter 310. The needle 104 and the catheter 310 are positioned adjacent a surface 500 of a vascular entity 502 to be punctured. In embodiments, the surface 500 may be the exterior surface 206, 210 and the interior surface 208, 212 of respective first and second vascular entities 200, 202, and the vascular entity may be one of the first and second vascular entities 200, 202. Rather than operating the firing device 102 to fire the needle 104 at the surface 500 of the vascular entity 502 to potentially slide away from the surface 500 upon contact, the suction device 304, such as the syringe 326 depicted in FIG. 7 or the external vacuum motor 402 depicted in FIG. 9, is operated as described herein to create a suction force within the suction channel 322 between the needle 104 and the catheter 310. The suction force being drawn through the suction channel 322 is depicted by arrows B.

Referring now to FIG. 11, the suction force draws a portion of the surface 500 of the vascular entity 502 toward the distal end 308 of the catheter 310. By drawing the portion of the surface 500 of the vascular entity 502 toward the distal end 308 end of the catheter 310, the surface 500 of the vascular entity 502 to be punctured by the needle 104 is at least partially retained at the distal end 308 of the catheter 310. This reduces the likelihood of potential slippage of the needle 104 against the surface 500 of the vascular entity 502 as the needle 104 is fired forward (e.g., distally) by the firing device 102 as described herein to puncture the surface 500 of the vascular entity 502.

Referring now to FIG. 12, the needle 104 is distally fired by the firing device 102 (FIG. 1) toward the surface 500 of the vascular entity 502 in the forward firing direction A1. As noted above, the suction force flowing within the suction channel 322 between the needle 104 and the catheter 310 causes the vascular entity 502 to hold its position relative to the distal end 308 of the catheter 310 as the needle 104 is fired. As such, this reduces the likelihood that the distal end 108 of the needle 104 will slip relative to the surface 500 of the vascular entity 502. Once the needle 104 punctures the surface 500 of the vascular entity 502, the suction device 304 may be deactivated so that the suction force is no longer present within the suction channel 322.

In embodiments, the suction device 304 may be repeatedly operated with each firing of the needle 104 by the firing device 102. As such, in embodiments, the suction device 304 may be operated either manually or automatically to create the suction force prior to the firing device 102 firing the needle 104 and deactivated after the firing device 102 fires the needle 104.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Directional terms as used herein—for example up, down, right, left, front, back, top, bottom—are made only with reference to the figures as drawn and are not intended to imply absolute orientation.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

As used herein, the singular forms “a,” “an” and “the” include plural referents unless the context clearly dictates otherwise. Thus, for example, reference to “a” component includes aspects having two or more such components, unless the context clearly indicates otherwise.

Embodiments may be further described with reference to the following numbered clauses:
1. A needle firing assembly comprising: a firing device; a catheter extending from the firing device; a needle extending from the firing device and through an interior of the catheter, the needle having a curvature proximate a distal end of the needle opposite the firing device; and a suction device in fluid communication with an interior of the catheter, the suction device configured to draw a suction force through a distal end of the catheter opposite the firing device, wherein the firing device is configured to move the needle in a forward firing direction relative to the catheter to extend the distal end of the needle through the distal end of the catheter.
2. The needle firing assembly of clause 1, wherein: the catheter defines a suction channel between an interior surface of the catheter and an exterior surface of the needle; the suction channel surrounds the needle; and the suction device is in fluid communication with the suction channel.
3. The needle firing assembly of any of clause 1 to clause 2, wherein the suction device is coupled to the catheter between the distal end of the catheter and the firing device.
4. The needle firing assembly of any of clause 1 to clause 3, wherein the suction device comprises a syringe.
5. The needle firing assembly of any of clause 1 to clause 4, wherein the suction device comprises an external vacuum motor.

6. The needle firing assembly of any of clauses 1-5, further comprising a puncture assist device provided at the distal end of the needle.
7. The needle firing assembly of clause 6, wherein the puncture assist device comprises a radiofrequency cutting device located at the distal end of the needle.
8. A needle firing assembly comprising: a firing device; a catheter extending from the firing device; a needle extending from the firing device and through an interior of the catheter; and a suction device in fluid communication with an interior of the catheter configured to draw fluid through a distal end of the catheter, wherein the firing device is configured to move the needle relative to the catheter to extend a distal end of the needle through the distal end of the catheter when in a firing state and retract the needle relative to the catheter such that the distal end of the needle is withdrawn from the distal end of the catheter when in a retracted state.
9. The needle firing assembly of clause 8, wherein: the needle has a curvature proximate a distal end of the needle opposite the firing device; the catheter defines a suction channel between an interior surface of the catheter and an exterior surface of the needle; the suction channel surrounds the needle; and the suction device is in fluid communication with the suction channel.
10. The needle firing assembly of any of clause 8 to clause 9, wherein the suction device is coupled to the catheter between the distal end of the catheter and the firing device.
11. The needle firing assembly of any of clause 8 to clause 10, wherein the suction device comprises a syringe.
12. The needle firing assembly of any of clause 8 to clause 10, wherein the suction device comprises an external vacuum motor.
13. The needle firing assembly of any of clauses 8-12, further comprising a puncture assist device provided at the distal end of the needle.
14. The needle firing assembly of clause 13, wherein the puncture assist device comprises a radiofrequency cutting device located at the distal end of the needle.
15. A method for puncturing a surface of a vascular entity, the method comprising: positioning a needle firing assembly relative to a surface of a vascular entity, the needle firing assembly comprising: a firing device; a catheter extending from the firing device; a needle extending from the firing device and through an interior of the catheter, the needle having a curvature proximate a distal end of the needle, a distal end of the catheter positioned at the surface of the vascular entity; and a suction device in fluid communication with an interior of the catheter; operating the suction device to draw fluid through the distal end of the catheter toward the firing device; creating a suction force drawing the surface of the vascular entity toward the distal end of the catheter; and operating the firing device to move the needle in a forward firing direction relative to the catheter to extend the distal end of the needle through the distal end of the catheter and puncture the surface of the vascular entity.
16. The method of clause 15, wherein: the catheter defines a suction channel between an interior surface of the catheter and an exterior surface of the needle; the suction channel surrounds the needle; and the suction device is in fluid communication with the suction channel.
17. The method of any of clause 15 to clause 16, wherein the suction device is coupled to the catheter between the distal end of the catheter and the firing device.
18. The method of any one of clauses 15-17, wherein the suction device comprises a syringe.
19. The method of any one of clause 15-17, wherein the suction device comprises an external vacuum motor.
20. The method of any one of clauses 15-19, wherein the needle firing assembly further comprises a puncture assist device provided at the distal end of the needle, and the puncture assist device comprises a radiofrequency cutting device located at the distal end of the needle.

From the above, it is to be appreciated that defined herein is a needle firing assembly 100, 300, 400 including a curved needle 104, 104A-104C that may be fired into adjacent vascular entities to facilitate placement of a stent between the vascular entities 200, 202, 502. To reduce the likelihood of a distal end of the needle 104, 104A-104C slipping against a surface of either vascular entity 200, 202, 502 during firing, the needle firing assembly 100, 300, 400 includes a suction device 304 configured to draw a suction force through a distal end 118, 308 of a catheter 110, 310 of the needle firing assembly 100, 300, 400, thereby drawing a portion of the surface of the vascular entities 200, 202, 502 toward the catheter 110, 310 and securing the vascular entity 200, 202, 502 to be punctured in position relative to the catheter 110, 310 prior to and during firing of the needle 104, 104A-104C.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A needle firing assembly comprising:
a firing device;
a catheter extending from the firing device;
a needle extending from the firing device and through an interior of the catheter, the needle having a curvature proximate a distal end of the needle opposite the firing device; and
a suction device in fluid communication with an interior of the catheter, the suction device configured to draw a suction force through a distal end of the catheter opposite the firing device,
wherein the firing device is configured to move the needle in a forward firing direction relative to the catheter to extend the distal end of the needle through the distal end of the catheter.

2. The needle firing assembly of claim 1, wherein:
the catheter defines a suction channel between an interior surface of the catheter and an exterior surface of the needle:
the suction channel surrounds the needle; and
the suction device is in fluid communication with the suction channel.

3. The needle firing assembly of claim 2, wherein the suction device is coupled to the catheter between the distal end of the catheter and the firing device.

4. The needle firing assembly of claim 3, wherein the suction device comprises a syringe.

5. The needle firing assembly of claim 3, wherein the suction device comprises an external vacuum motor.

6. The needle firing assembly of claim 1, further comprising a puncture assist device provided at the distal end of the needle.

7. The needle firing assembly of claim 6, wherein the puncture assist device comprises a radiofrequency cutting device located at the distal end of the needle.

8. A needle firing assembly comprising:
a firing device;
a catheter extending from the firing device;
a needle extending from the firing device and through an interior of the catheter; and
a suction device in fluid communication with an interior of the catheter configured to draw fluid through a distal end of the catheter,
wherein the firing device is configured to move the needle relative to the catheter to extend a distal end of the needle through the distal end of the catheter when in a firing state and retract the needle relative to the catheter such that the distal end of the needle is withdrawn from the distal end of the catheter when in a retracted state.

9. The needle firing assembly of claim 8, wherein:
the needle has a curvature proximate a distal end of the needle opposite the firing device;
the catheter defines a suction channel between an interior surface of the catheter and an exterior surface of the needle:
the suction channel surrounds the needle; and
the suction device is in fluid communication with the suction channel.

10. The needle firing assembly of claim 9, wherein the suction device is coupled to the catheter between the distal end of the catheter and the firing device.

11. The needle firing assembly of claim 10, wherein the suction device comprises a syringe.

12. The needle firing assembly of claim 10, wherein the suction device comprises an external vacuum motor.

13. The needle firing assembly of claim 8, further comprising a puncture assist device provided at the distal end of the needle.

14. The needle firing assembly of claim 13, wherein the puncture assist device comprises a radiofrequency cutting device located at the distal end of the needle.

15. A method for puncturing a surface of a vascular entity, the method comprising:
positioning a needle firing assembly relative to a surface of a vascular entity, the needle firing assembly comprising:
a firing device;
a catheter extending from the firing device;
a needle extending from the firing device and through an interior of the catheter, the needle having a curvature proximate a distal end of the needle, a distal end of the catheter positioned at the surface of the vascular entity; and
a suction device in fluid communication with an interior of the catheter;
operating the suction device to draw fluid through the distal end of the catheter toward the firing device;
creating a suction force drawing the surface of the vascular entity toward the distal end of the catheter; and
operating the firing device to move the needle in a forward firing direction relative to the catheter to extend the distal end of the needle through the distal end of the catheter and puncture the surface of the vascular entity.

16. The method of claim 15, wherein:
the catheter defines a suction channel between an interior surface of the catheter and an exterior surface of the needle:
the suction channel surrounds the needle; and
the suction device is in fluid communication with the suction channel.

17. The method of claim 16, wherein the suction device is coupled to the catheter between the distal end of the catheter and the firing device.

18. The method of claim 16, wherein the suction device comprises a syringe.

19. The method of claim 16, wherein the suction device comprises an external vacuum motor.

20. The method of claim 16, wherein the needle firing assembly further comprises a puncture assist device provided at the distal end of the needle, and the puncture assist device comprises a radiofrequency cutting device located at the distal end of the needle.
